(19) United States
(12) Patent Application Publication
(10) Pub. No.: US 2026/0069818 A1
(43) Pub. Date: Mar. 12, 2026

(54) VISUAL BRAIN THERAPY DEVICE

(71) Applicant: Anthony Lemmo, LaSalle (CA)

(72) Inventor: Anthony Lemmo, LaSalle (CA)

(21) Appl. No.: 18/707,059

(22) PCT Filed: Nov. 2, 2022
(86) PCT No.: PCT/CA2022/051617

§ 371 Date: May 2, 2024

Related U.S. Application Data
(60) Provisional application No. 63/274,570, filed on Nov. 2, 2021.

(51) Int. Cl. A61M 21/00

(52) U.S. Cl. CPC A61M 21/00; A61M 2021/0044

(57) ABSTRACT

A visual brain therapy device comprises a vertical panel, a horizontal panel, a plurality of lights and a controller. The vertical panel has an aperture, where the aperture has an upper edge and an opposite lower edge. The horizontal panel has a front end and an opposite back end, where the front end of the horizontal panel aligns with the lower edge of the aperture. Each of the plurality of lights is mounted on either the vertical panel or the horizontal panel. The controller is configured to activate each of the plurality of lights in a pattern, where the pattern comprises one of convergence, divergence, saccades and anti-saccades.

### CROSS-REFERENCE TO RELATED APPLICATIONS

This application claims priority to U.S. provisional patent application no. 63/274,570 , filed on Nov. 2, 2021, the disclosure of which is incorporated herein by reference.

### TECHNICAL FIELD

The present invention relates to a device that provides visual stimuli to allow a patient to perform various eye movement exercises to build and/or strengthen neural pathways in the brain.

### BACKGROUND OF THE INVENTION

Functional neurology eye movement training is an effective method for brain rehabilitation to promote neuroplastic changes. Not only can it be used to treat brain injuries caused by strokes, sports injuries or car accidents, and various brain function impairments, such as dementia, attention deficit disorder (“ADD”) or attention deficit hyperactivity disorder (“ADHD”), but it also may be used for performance enhancement.

To build and/or strengthen neural pathways in the brain, a patient is prescribed various eye exercises where the patient follows an object or a series of objects in a specific pattern and at specific speeds in order to stimulate specific areas of the brain. Typically, the objects are provided on a two-dimensional surface, such as on an iPad or on a sheet of paper. Thus, conventional eye movement exercises do not fully mimic real-life eye movements because they do not include a depth component.

Moreover, there are a number of ways in which conventional eye movement exercises fail to create a consistent, repeatable and accurate method for performing eye exercise. First, with conventional eye movement exercises, it is difficult to control the spatial orientation of the screen relative to the patient. For example, it is difficult to control the distance between the iPad and the patient's eyes or the orientation of the iPad in the vertical or horizontal plane relative to the patient's eyes. This also makes the object movements imprecise and difficult to repeat consistently.

Second, with conventional eye movement exercises, it is difficult to isolate eye movement apart from head movement. For the process to work properly, it is important that the patient follows the object(s) only with his or her eyes without moving his or her head. But E with conventional eye movement exercises, there is no way to immobilize the patient's head to ensure that the patient is tracking the object only with his or her eyes.

### SUMMARY OF THE INVENTION

According to one embodiment, there is provided a visual brain therapy device comprises a vertical panel, a horizontal panel, a plurality of lights and a controller. The vertical panel has an aperture, where the aperture has an upper edge and an opposite lower edge. The horizontal panel has a front end and an opposite back end, where the front end of the horizontal panel aligns with the lower edge of the aperture. Each of the plurality of lights is mounted on either the vertical panel or the horizontal panel. The controller is configured to activate each of the plurality of lights in a pattern, where the pattern comprises one of convergence, divergence, saccades and anti-saccades.

According to another embodiment, there is a provided a visual brain therapy device comprises a vertical panel, a horizontal panel, a plurality of lights and a controller. The vertical panel has an aperture, where the aperture has an upper edge and an opposite lower edge. The horizontal panel has a front end, an opposite back end, a left end and an opposite right end. The front end of the horizontal panel aligns with the lower edge of the aperture. Each of the plurality of lights is mounted on either the vertical panel or the horizontal panel, where one of the plurality of lights is mounted on the horizontal panel closer to the front end and the left end than a second of the plurality of lights. The controller is configured to activate each of the plurality of lights.

### BRIEF DESCRIPTION OF THE DRAWINGS

Advantages of the present invention will be readily appreciated as the same becomes better understood by reference to the following detailed description when considered in connection with the accompanying drawings wherein:

FIG. 1 is a perspective view of a visual brain therapy device in accordance with one embodiment of the present invention;

FIG. 2 is a front view of the visual brain therapy device of FIG. 1;

FIG. 3 is a side view of the visual brain therapy device of FIG. 1;

FIG. 4 is a perspective view of the visual brain therapy device of FIG. 1 with the arm on the head and chin rest extended;

FIG. 5 is a front view of the front panel on the visual brain therapy device housing of FIG. 1;

FIG. 6 is a top view of the inner panel in the visual brain therapy device of FIG. 1; and

FIG. 7 shows one embodiment of a control panel for the visual brain therapy device of FIG. 1.

### DETAILED DESCRIPTION OF EXAMPLE EMBODIMENTS

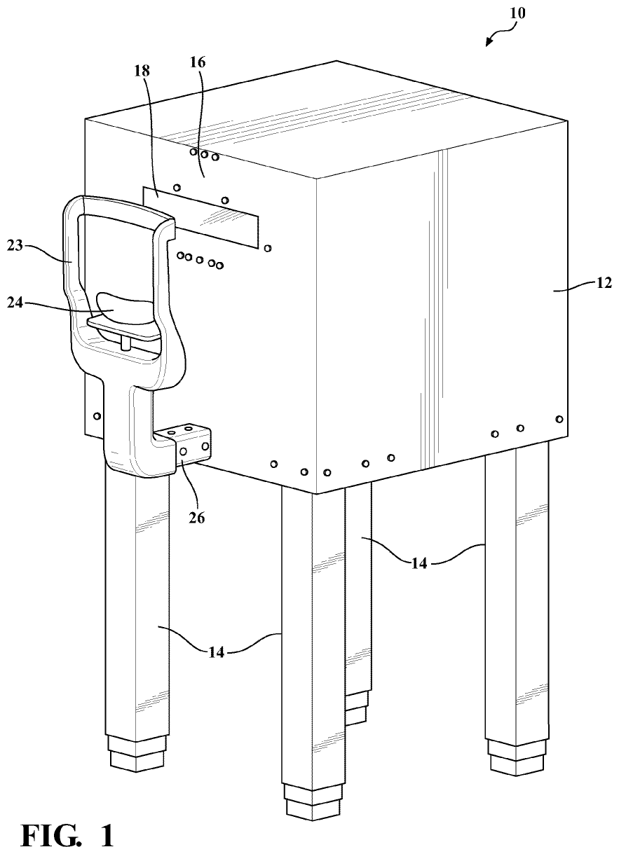

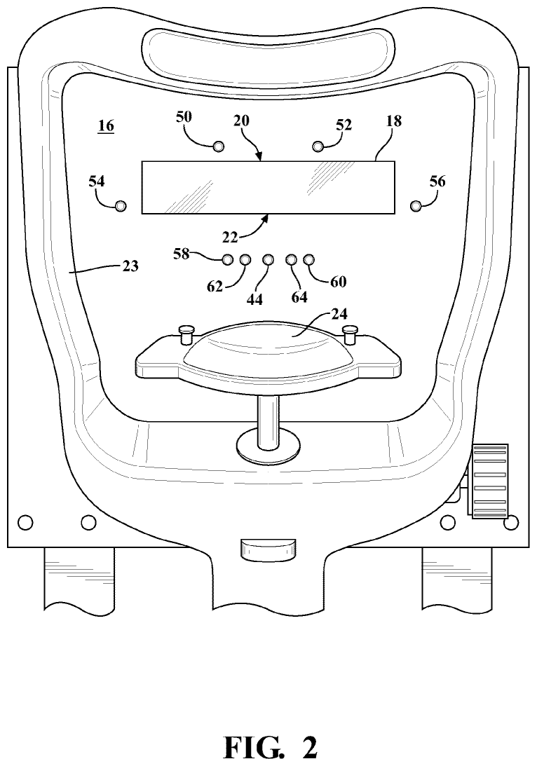

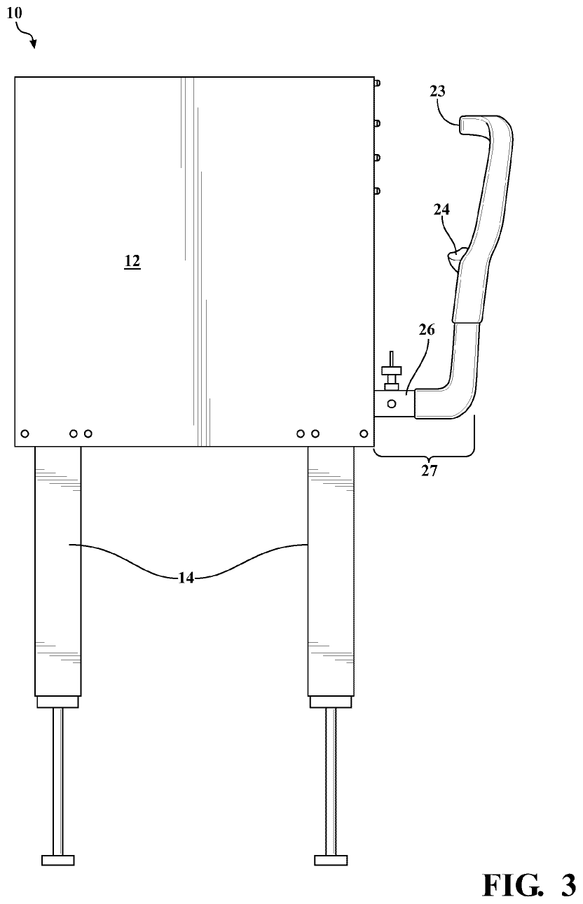

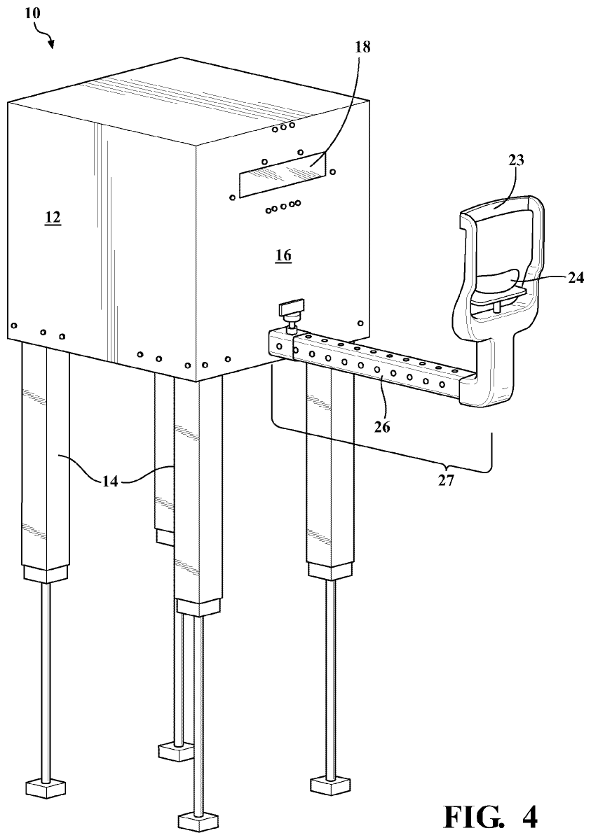

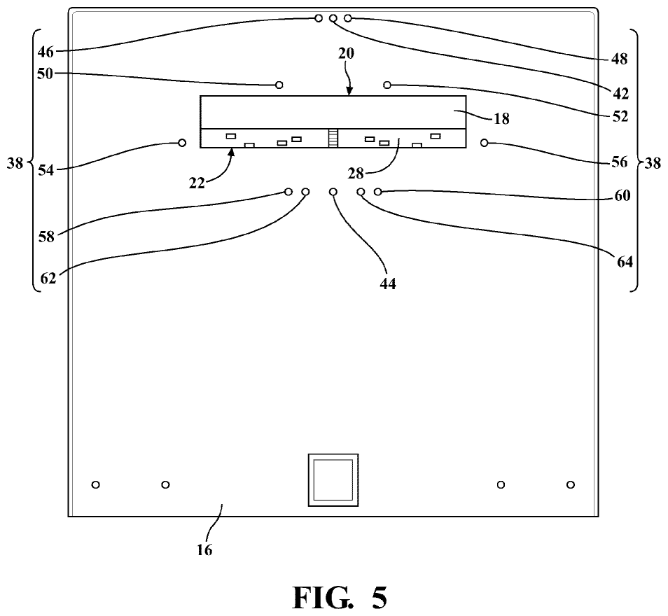

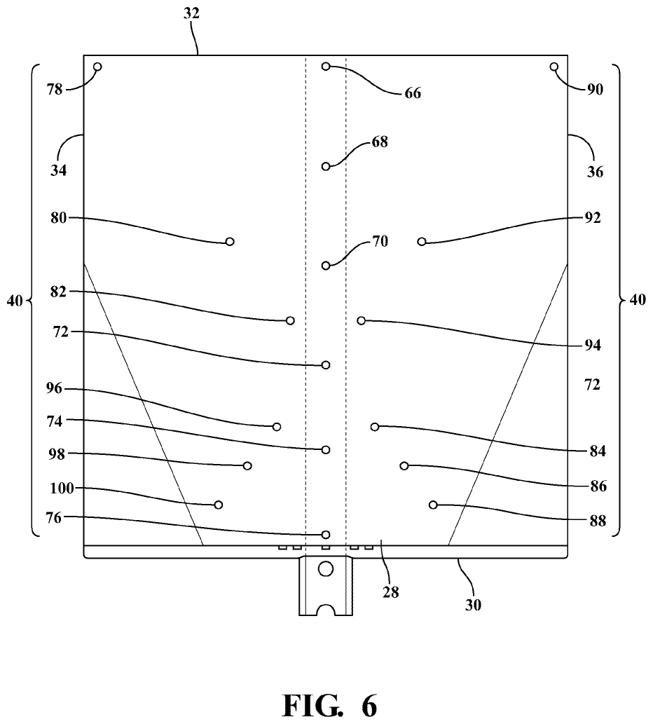

FIGS. 1-4 illustrate one embodiment of a visual brain therapy device 10 of the present invention. The visual brain therapy device 10 includes a housing 12 mounted on legs 14. A front panel 16 of the housing 12 includes an aperture 18 having an upper edge 20 and an opposite lower edge 22. A head 23 and chin rest 24 are mounted on an extendable arm 26 extending from the front panel 16 of the housing 12. The chin rest 24 may be raised or lowered to ensure that the patients' eyes are aligned with the aperture 18 on the front panel 16 of the housing 12. Referring to FIG. 4, the arm 26 has a length 27 which is adjustable to ensure that the patient is at a proper distance away from the device 10. Referring to FIGS. 5 and 6, an inner panel 28 is mounted in a horizontal plane within the housing 12, and is aligned with the lower edge 22 of the aperture 18. The inner panel 28 includes a front end 30, a rear end 32, a left end 34, and a right end 36. A plurality of lights 38 are mounted on the front panel 16, and a plurality of lights 40 are mounted on the inner panel 28.

Referring to FIG. 5, the front panel 16 of the device 10 includes five lights 38 above the aperture 18, five lights 38 below the aperture 18, and one light 38 on each side of the aperture 18. An upper vertical light 42 is mounted above the center of the aperture 18, and a lower vertical light 44 is mounted below the center of the aperture 18. The remainder of the lights 38 on the front panel 16 stimulate specific eye muscles. In particular, light 46 stimulates the left inferior oblique, light 48 stimulates the right inferior oblique, light 50 stimulates the left superior rectus, light 52 stimulates the right superior rectus, light 54 stimulates the left lateral rectus, light 56 stimulates the right lateral rectus, light 58 stimulates the left inferior rectus, light 60 stimulates the right inferior rectus, light 62 stimulates the left superior oblique, and light 64 stimulates the right superior oblique.

Referring to FIG. 6, the inner panel 28 includes six lights 66, 68, 70, 72, 74, 76 mounted along the center of the panel 28 from the rear 32 to the front 30 of the panel 28. The inner panel 28 also includes six lights 78, 80, 82, 84, 86, 88 extending along a left-right diagonal from the rear 32 to the front 30 of the inner panel 28. The fourth center light 72 lies between the third 82 and fourth 84 left-right diagonal lights. The inner panel 28 also includes six lights 90, 92, 94, 96, 98, 100 extending along a right-left diagonal from the rear 32 to the front 30 of the panel 28. The fourth center light 72 lies between the third 94 and fourth 96 right-left diagonal lights. Because the inner panel 28 lies in the horizontal plane, the visual brain therapy device 10 provides a plurality of lights in three-dimensions, thereby integrating both depth and distance of the lights in such a way that makes eye exercises uniform and more effective.

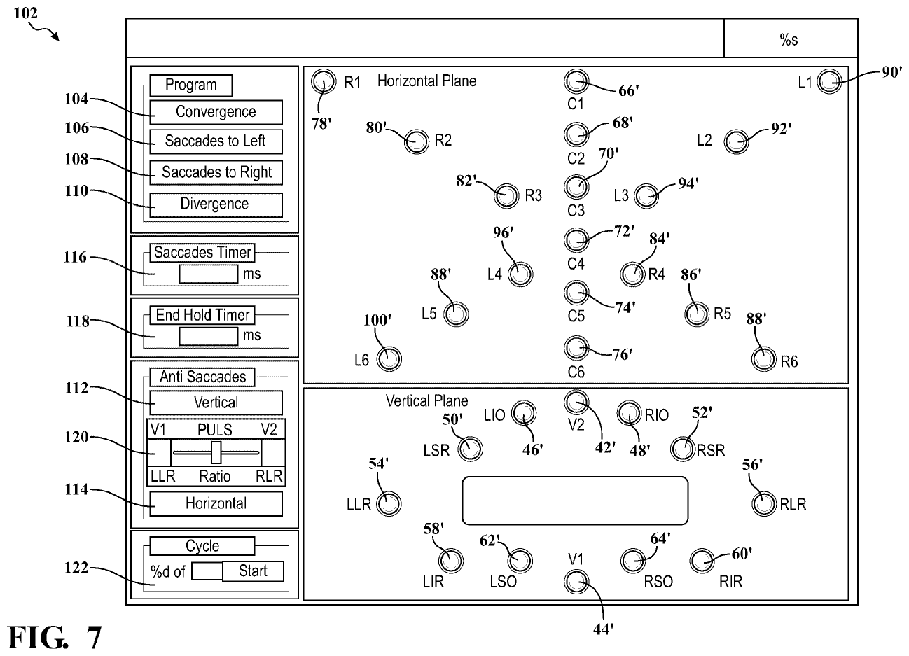

FIG. 7 shows one embodiment of a control panel 102 for the visual brain therapy device 10. The control panel 102 includes lights that correspond to the lights 38 on the front panel 16, and lights 40 on the inner panel 28 so that the operator of the device 10 may monitor the light sequence on the device 10. For ease of reference, the corresponding lights on the control panel 102 are represented by like primed reference numerals.

The control panel 102 includes a plurality of eye exercises for the visual brain therapy device 10 including convergence 104, saccades to the left 106, saccades to the right 108, divergence 110, vertical anti-saccades 112, and horizontal anti-saccades 114. With convergence 104, a patient tracks a series of lights as they approach toward the patient, and with divergence 110, a patient tracks a series of light as they move away from the patient. Saccades are rapid, ballistic movements of the eyes. Saccades to the left 106 require the patient to track a series of lights as they move along the right-left diagonal from the rear to the front of the device 10, and saccades to the right 108 require the patient to track a series of lights as they move along the left-right diagonal from the rear to the front of the device 10. Vertical anti-saccades 112 require the patient to make a saccadic eye movement away from a target in the vertical direction, and horizontal anti-saccades require the patient to make a saccadic eye movement away from a target in the horizontal direction. The visual brain therapy device 10 also may be programed to test gaze holding.

The control panel 102 also includes a saccades timer 116 and an end hold timer 118 to allow the operator to control the speed and hold time of the lights 38, 40 during the various exercises. The control panel 102 allows the operator to control the anti-saccades pulse ratio 120, and allows the operator to control the number of cycles 122 performed for each exercise.

In operation, the operator programs the eye exercises into the visual brain therapy device 10 using the control panel 102. For example, to program convergence at a speed of 1 second with a hold time of 2 seconds, the operator selects the convergence option 104, sets the saccades timer 116 to 1000 ms and the end hold timer 118 to 2000 ms. After selecting the number of cycles 122, the operator activates the program. The device 10 will (1) turn on light 66 for one second, (2) turn off light 66 and turn on light 68 for one second, (3) turn off light 68 and turn on light 70 for one second, (4) turn off light 70 and turn on light 72 for one second, (5) turn off light 72 and turn on light 74 for one second, (6) turn off light 74 and turn on light 76 for one second, (7) turn off light 76 and (8) wait 2 seconds before repeating the process for as many cycles as programmed by the operator. The patient is instructed to follow the light pattern with his or her eyes as the lights approach toward the patient.

Similarly, to program divergence at a speed of 1 second with a hold time of 2 seconds, the operator selects the divergence option 110, sets the saccades timer 116 to 1000 ms and the end hold timer 118 to 2000 ms. After selecting the number of cycles 122, and activating the program, the device 10 will (1) turn on light 76 for one second, (2) turn off light 76 and turn on light 74 for one second, (3) turn off light 74 and turn on light 72 for one second, (4) turn off light 72 and turn on light 70 for one second, (5) turn off light 70 and turn on light 68 for one second, (6) turn off light 68 and turn on light 66 for one second, (7) turn off light 66 and (8) wait 2 seconds before repeating the process for as many cycles as programmed by the operator. The patient is instructed to follow the light pattern with his or her eyes as the lights move away from the patient.

To program saccades to the left at a speed of 0.5 seconds with a hold time of 2 seconds with a final right lateral rectus, the operator selects the saccades to the left option 106, sets the saccades timer 116 to 500 ms and the end hold timer 118 to 2000 ms. After selecting the number of cycles 122 and activating the program, the device 10 will (1) turn on light 90 for 0.5 seconds, (2) turn off light 90 and turn on light 92 for 0.5 seconds, (3) turn off light 92 and turn on light 94 for 0.5 seconds, (4) turn off light 94 and turn on light 72 for 0.5 seconds, (5) turn off light 72 and turn on light 96 for 0.5 seconds, (6) turn off light 96 and turn on light 98 for 0.5 seconds, (7) turn off light 98 and turn on light 100 for 0.5 seconds, (8) turn off light 100 and turn on light 54 for 2 seconds, (9) turn off light 54 and turn on light 56 for 2 seconds, (10) turn off light 56 and (11) wait 2 seconds before repeating the process for as many cycles as programmed by the operator. The patient is instructed to follow the light pattern with his or her eyes throughout the exercise.

To program saccades to the right at a speed of 0.5 seconds with a hold time of 2 seconds with a final left lateral rectus, the operator selects the saccades to the right option 108, sets the saccades timer 116 to 500 ms and the end hold timer 118 to 2000 ms. After selecting the number of cycles 122 and activating the program, the device 10 will (1) turn on light 78 for 0.5 seconds, (2) turn off light 78 and turn on light 80 for 0.5 seconds, (3) turn off light 80 and turn on light 82 for 0.5 seconds, (4) turn off light 82 and turn on light 72 for 0.5 seconds, (5) turn off light 72 and turn on light 84 for 0.5 seconds, (6) turn off light 84 and turn on light 86 for 0.5 seconds, (7) turn off light 86 and turn on light 88 for 0.5 seconds, (8) turn off light 88 and turn on light 56 for 2 seconds, (9) turn off light 56 and turn on light 54 for 2 seconds, (10) turn off light 54 and (11) wait 2 seconds before repeating the process for as many cycles as programmed by the operator. The patient is instructed to follow the light pattern with his or her eyes throughout the exercise.

To program vertical anti-saccades at a speed of 0.5 seconds with a hold time of 2 seconds, the operator selects the vertical anti-saccades option 112, sets the saccades timer 116 to 500 ms, sets the end hold timer 118 to 2000 ms, and sets the anti-saccades pulse ratio 120. After selecting the number of cycles 122 and activating the program, the device 10 will initially turn on the front center light 76 on the inner panel 28, and the upper vertical light 42 and the lower vertical light 44 on the front panel 16. Then the device 10 will turn off the upper vertical light 42 and the lower vertical light 44 and begin the anti-saccades sequence. During the sequence, either the upper vertical light 42 or the lower vertical light 44 will blink for a period of 0.5 seconds, and the patient is instructed to look at the light that is not blinking. Thus, if the upper vertical light 42 blinks during the cycle, then the patients should look at lower vertical light 44. Alternatively, if the lower vertical light 44 blinks during the cycle, then the patient should look at the upper vertical light 42. The light 42, 44 that blinks will be random, but the number of times that the upper vertical light 42 blinks versus the number of times that the lower vertical light 44 blinks depends on the anti-saccades pulse ratio 120 and the number of cycles 122 selected. If the ratio 120 is 50:50 and the number of cycles 122 is set to 10, then the lower vertical light 44 will randomly blink 5 times and the upper vertical light 42 will randomly blink 5 times during the exercise. If, on the other hand, the ratio 120 is 30:70 and the number of cycles 122 is set to 10, then the lower vertical light 44 will randomly blink 3 times and the upper vertical light 42 will randomly blink 7 times during the exercise. There is a 2 second wait period between each cycle when neither the upper vertical light 42 nor the lower vertical light 44 blinks.

To program horizontal anti-saccades at a speed of 0.5 seconds with a hold time of 2 seconds, the operator selects the horizontal anti-saccades option 114, sets the saccades timer 116 to 500 ms, sets the end hold timer 118 to 2000 ms, and sets the anti-saccades pulse ratio 120. After selecting the number of cycles 122 and activating the program, the device 10 will initially turn on the front center light 76 on the inner panel 28, and the left lateral rectus light 54 and the right lateral rectus light 56 on the front panel 16. Then the device 10 will turn off the left lateral rectus light 54 and the right lateral rectus light 56 and begin the anti-saccades sequence. During the sequence, either the left lateral rectus light 54 or the right lateral rectus light 56 will blink for a period of 0.5 seconds, and the patient is instructed to look at the light that is not blinking. Thus, if the left lateral rectus light 54 blinks during the cycle, then the patients should look at the right lateral rectus light 56. Alternatively, if the right lateral rectus light 56 blinks during the cycle, then the patient should look at the left lateral rectus light 54. The light 54, 56 that blinks will be random, but the number of times that the left lateral rectus light 54 blinks versus the number of times that the right lateral rectus light 56 blinks depends on the anti-saccades pulse ratio 120 and the number of cycles 122 selected. If the ratio 120 is 50:50 and the number of cycles 122 is set to 10, then the left lateral rectus light 54 will randomly blink 5 times and the right lateral rectus light 56 will randomly blink 5 times during the exercise. If, on the other hand, the ratio 120 is 30:70 and the number of cycles 122 is set to 10, then the left lateral rectus light 54 will randomly blink 3 times and the right lateral rectus light 56 will randomly blink 7 times during the exercise. There is a 2 second wait period between each cycle when neither the left lateral rectus light 54 nor the right lateral rectus light 56 blinks.

The invention has been described in an illustrative manner, and it is to be understood that the terminology, which has been used, is intended to be in the nature of words of description rather than of limitation. Many modifications and variations of the present invention are possible in light of the above teachings. It is, therefore, to be understood that within the scope of the appended claims, the invention may be practiced other than as specifically described.

1. A visual brain therapy device comprising:
a vertical panel having an aperture, wherein the aperture has an upper edge and an opposite lower edge;
a horizontal panel having a front end and an opposite back end, wherein the front end of the horizontal panel aligns with the lower edge of the aperture;
a plurality of lights, wherein each of the plurality of lights is mounted on either the vertical panel or the horizontal panel; and
a controller configured to activate each of the plurality of lights in a pattern, wherein the pattern comprises one of convergence, divergence, saccades and anti-saccades.

2. The visual brain therapy device of claim 1, wherein the controller is configured to adjust a speed at which the plurality of lights are activated.

3. The visual brain therapy device of claim 1, wherein the controller is configured to adjust a hold time during which each of the plurality of lights are activated.

4. The visual brain therapy device of claim 1, wherein the controller is configured to adjust a number of times the pattern is repeated.

5. The visual brain therapy device of claim 1, wherein the controller is configured to adjust a pulse ratio for the anti-saccades.

6. The visual brain therapy device of claim 1, wherein the saccades comprises saccades to the left or saccades to the right.

7. The visual brain therapy device of claim 1, wherein the anti-saccades comprises vertical anti-saccades or horizontal anti-saccades.

8. The visual brain therapy device of claim 1, further comprising:
a chin rest; and
an arm having a first end mounted to the vertical panel and a second end mounted to the chin rest, wherein a length between the first end and the second end is adjustable.

9. A visual brain therapy device comprising:
a vertical panel having an aperture, wherein the aperture has an upper edge and an opposite lower edge;
a horizontal panel having a front end, an opposite back end, a left end and an opposite right end, wherein the front end of the horizontal panel aligns with the lower edge of the aperture;
a plurality of lights, wherein each of the plurality of lights is mounted on either the vertical panel or the horizontal panel, wherein one of the plurality of lights is mounted on the horizontal panel closer to the front end and the left end than a second of the plurality of lights; and
a controller configured to activate each of the plurality of lights.

10. The visual brain therapy device of claim 9, wherein the controller is configured to activate each of the plurality of lights in a pattern, wherein the pattern comprises one of convergence, divergence, saccades and anti-saccades.

11. The visual brain therapy device of claim 10, wherein the controller is configured to adjust a speed at which the plurality of lights are activated.

12. The visual brain therapy device of claim 10, wherein the controller is configured to adjust a hold time during which each of the plurality of lights are activated.

13. The visual brain therapy device of claim 10, wherein the controller is configured to adjust a number of times the pattern is repeated.

14. The visual brain therapy device of claim 10, wherein the controller is configured to adjust a pulse ratio for the anti-saccades.

15. The visual brain therapy device of claim 10, wherein the saccades comprises saccades to the left or saccades to the right.

16. The visual brain therapy device of claim 10, wherein the anti-saccades comprises vertical anti-saccades or horizontal anti-saccades.

17. The visual brain therapy device of claim 9, further comprising:
a chin rest; and
an arm having a first end mounted to the vertical panel and a second end mounted to the chin rest, wherein a length between the first end and the second end is adjustable.
